(19) United States
(12) Patent Application Publication
(10) Pub. No.: US 2009/0099065 A1
(43) Pub. Date: Apr. 16, 2009

(54) Acylated Single Chain Insulin

(75) Inventors: Peter Madsen, Bagsvaerd (DK); Thomas Borglum Kjeldsen, Virum (DK); Tina Moller Tagmose, Ballerup (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 12/282,788

(22) PCT Filed: Mar. 12, 2007
(86) PCT No.: PCT/EP2007/052294

§ 371 Date: Oct. 24, 2008

(30) Foreign Application Priority Data
Mar. 13, 2006 (EP) 06111022.7

(51) Int. Cl. A61K 38/28; C07K 14/62

(52) U.S. Cl. 514/3; 530/303

(57) ABSTRACT

The invention is related to an acylated, single-chain insulin comprising the B- and the A-chain of human insulin or an analogue thereof connected by a connecting peptide, wherein a lysine residue being substituted for the natural amino acid residue in one of the positions A12-A23 in the human insulin A-chain has been chemically modified by acylation.

### FIELD OF THE INVENTION

The present invention is related to acylated, single-chain insulins and to pharmaceutical compositions comprising such acylated, single-chain insulins.

### BACKGROUND OF THE INVENTION

Currently, the treatment of diabetes, both type 1 diabetes and type 2 diabetes, relies to an increasing extent on the so-called intensive insulin treatment. According to this regimen, the patients are treated with multiple daily insulin injections comprising one or two daily injections of a long acting insulin to cover the basal insulin requirement supplemented by bolus injections of a rapid acting insulin to cover the insulin requirement related to meals.

Long acting insulin compositions are well known in the art. Thus, one main type of long acting insulin compositions comprises injectable aqueous suspensions of insulin crystals or amorphous insulin. In these compositions, the insulin compounds utilized typically are protamine insulin, zinc insulin or protamine zinc insulin.

Certain drawbacks are associated with the use of insulin suspensions. Thus, in order to secure an accurate dosing, the insulin particles must be suspended homogeneously by gentle shaking before a defined volume of the suspension is withdrawn from a vial or expelled from a cartridge. Also, for the storage of insulin suspensions, the temperature must be kept within more narrow limits than for insulin solutions in order to avoid lump formation or coagulation.

Another type of long acting insulin compositions are solutions having a pH value below physiological pH from which the insulin will precipitate because of the rise in the pH value when the solution is injected. A drawback with these solutions is that the particle size distribution of the precipitate formed in the tissue on injection, and thus the release profile of the medication, depends on the blood flow at the injection site and other parameters in a somewhat unpredictable manner. A further drawback is that the solid particles of the insulin may act as a local irritant causing inflammation of the tissue at the site of injection.

A further group of long acting or protracted insulin derivates are acylated insulin derivates. Human insulin has three primary amino groups: the N-terminal group of the A-chain and of the B-chain and the ε-amino group of the lysine group in position B29 in the B-chain. Soluble insulin derivatives containing lipophilic substituents linked to the ε-amino group of a lysine residue in any of the positions B26 to B30 are disclosed in e.g. WO 95/07931, WO 96/00107, WO 97/31022, WO 2005/012347 and EP 894095. These insulin two-chain insulin derivatives have a prolonged profile of action and are soluble at physiological pH values.

Insulin is a polypeptide hormone secreted by β-cells of the pancreas and consists of two polypeptide chains, A and B, which are linked by two inter-chain disulphide bridges. Furthermore, the A-chain features one intra-chain disulphide bridge.

The hormone is synthesized as a single-chain precursor of proinsulin (preproinsulin) consisting of a prepeptide of 24 amino acid followed by proinsulin containing 86 amino acids in the configuration: prepeptide B-Arg Arg-C-Lys Arg-A, in which C is a connecting peptide of 31 amino acids. Arg-Arg and Lys-Arg are cleavage sites for cleavage of the connecting peptide from the A and B chains to form the two-chain insulin molecule. Insulin is essential in maintaining normal metabolic regulation.

The two chain structure of insulin allows insulin to undertake multiple conformations, and several findings have indicated that insulin has the propensity to considerable conformational change and that restrictions in the potential for such change considerably decrease the affinity for the insulin receptor. Proinsulin has a 100 fold lower affinity for the insulin receptor than native insulin.

On the other hand the more rigid structure of the un-cleaved, single chain insulin molecule may impart an increased physical and chemical stability to the insulin molecule. Physical and chemical stability are fundamental for insulin formulation and for applicable insulin administration methods, as well as for shelf-life and storage conditions of pharmaceutical preparations. Use of solutions in administration of insulin exposes the molecule to a combination of factors, e.g. elevated temperature, variable air-liquid-solid interphases as well as shear forces, which may result in irreversible conformation changes e.g. fibrillation. Thus physical and chemical stability of the insulin molecule is a basic condition for insulin therapy of diabetes mellitus.

Single-chain insulins having improved stability and at the same time an insulin activity comparable with human insulin have recently been disclosed in WO 2005/054291. Other types of single-chain insulins are disclosed in EP 1,193,272, EP 741,188, WO 95/16708 and WO 2005/054291. These single-chain insulins are characterized in having certain modified C-peptides with from 5-18, from 10-14 or from 5-11 amino acids residues in the modified Cpeptide. WO 2005/054291 further suggests to make the single-chain insulin protracted by acylating the parent single-chain insulin molecule.

It is the object of the present invention to provide a selected group of acylated single-chain insulin with improved properties over the known compounds both with respect to insulin activity, physical stability and solubility as well as a protracted action profile.

### SUMMARY OF THE INVENTION

In one aspect the present invention is related to an acylated, single-chain insulin comprising the B- and the A-chain of human insulin or an analogue thereof connected by a connecting peptide, wherein a lysine residue being substituted for the natural amino acid residue in one of the positions A12-A23 is chemically modified by acylation.

In another embodiment the invention is related to an acylated, single-chain insulin according being acylated at a lysine amino acid residue in position A12, A14, A15, A17, A18, A21, A22 or A23 in the human insulin A-chain.

In another embodiment the invention is related to an acylated, single-chain insulin being acylated at a lysine amino acid residue in position A18, A21, A22 or A23 in the human insulin A-chain.

In a further embodiment the invention is related to an acylated, single-chain insulin being acylated at a lysine amino acid residue in position A18 in the human insulin A-chain.

In a further embodiment the invention is related to an acylated, single-chain insulin being acylated at a lysine amino acid residue in position A22 in the human insulin A-chain.

The acylated, single chain insulin according to the present invention may also be acylated in the natural lysine amino acid residue in position B29 in the B-chain.

If, however, acylation of the natural lysine group in position B29 in the human insulin B-chain is unwanted this amino acid residue may be replaced by another amino acid residue. Suitable replacement amino acid residues are Ala, Arg, Gln and His. Alternatively the lysine amino acid residue in position B29 may be blocked by well known technology before acylation of the lysine residue in the desired position A8, A9 or A10 of the A-chain of insulin followed by deblocking after acylation in the desired position.

In a further embodiment, the amino acid residue in position B30 is deleted.

In one embodiment the acyl group is a lipophilic group derived from a fatty acid moiety having from about 6 to about 32 carbon atoms.

In another embodiment the fatty acid moiety have from 6 to 24, from 8 to 20, from 12 to 20, from 12-16, from 10-16, from 10-20, from 14-18 or from 14-16 carbon atoms.

The acyl group may either via an amide bond be directly attached to the ε-amino group of the lysine group in question. It may also be attached the lysine group via a linker group which via amide bond link the acyl group and the parent insulin molecule together.

In one embodiment the acyl group is connected to the lysine residue using an amino acid linker such as a γ- or an α-glutamyl linker, or via a β- or an α-aspartyl linker, or via an α-amido-γ-glutamyl linker, or via an α-amido-β-aspartyl linker.

The length of the connecting peptide may vary from 3 amino acid residues and up to a length corresponding to the length of the natural C-peptide in human insulin. The connecting peptide in the acylated, single-chain insulins according to the present invention is however normally shorter than the human C-peptide and will typically have a length from 3 to about 35, from 3 to about 30, from 4 to about 35, from 4 to about 30, from 5 to about 35, from 5 to about 30, from 6 to about 35 or from 6 to about 30, from 3 to about 25, from 3 to about 20, from 4 to about 25, from 4 to about 20, from 5 to about 25, from 5 to about 20, from 6 to about 25 or from 6 to about 20, from 3 to about 15, from 3 to about 10, from 4 to about 15, from 4 to about 10, from 5 to about 15, from 5 to about 10, from 6 to about 15 or from 6 to about 10, or from 6-9, 6-8, 6-7, 7-8, 7-9, or 7-10 amino acid residues in the peptide chain.

Non-limiting examples of useful connecting peptides are the sequences: VGLSSGQ (SEQ ID NO:1) and TGLGSGR (SEQ ID NO:2).

In still a further aspect the present invention is related to pharmaceutical preparations comprising the acylated, single-chain insulin of the invention and suitable adjuvants and additives such as one or more agents suitable for stabilization, preservation or isotoni, for example, zinc ions, phenol, cresol, a parabene, sodium chloride, glycerol or mannitol. The zinc content of the present formulations may be between 0 and about 6 zinc atoms per insulin hexamer. The pH of the pharmaceutical preparation may be between about 4 and about 8.5, between about 4 and about 5 or between about 6.5 and about 7.5.

In a further embodiment the present invention is related to the use of the acylated, single-chain insulin as a pharmaceutical for the reducing of blood glucose levels in mammalians, in particularly for the treatment of diabetes.

In a further aspect the present invention is related to the use of the acylated, single-chain insulin for the preparation of a pharmaceutical preparation for the reducing of blood glucose level in mammalians, in particularly for the treatment of diabetes.

In a further embodiment the present invention is related to a method of reducing the blood glucose level in mammalians by administrating a therapeutically active dose of an acylated, single-chain insulin according to the invention to a patient in need of such treatment.

In a further aspect of the present invention the acylated, single-chain insulins are administered in combination with one or more further active substances in any suitable ratios. Such further active agents may be selected from human insulin, fast acting insulin analogues, antidiabetic agents, antihyperlipidemic agents, antiobesity agents, antihypertensive agents and agents for the treatment of complications resulting from or associated with diabetes.

In one embodiment the two active components are administered as a mixed pharmaceutical preparation. In another embodiment the two components are administered separately either simultaneously or sequentially.

In one embodiment the acylated, single-chain insulins of the invention may be administered together with fast acting human insulin or human insulin analogues. Such fast acting insulin analogue may be such wherein the amino acid residue in position B28 is Asp, Lys, Leu, Val, or Ala and the amino acid residue in position B29 is Lys or Pro, des(B28-B30), des(B27) or des(B30) human insulin, and an analogue wherein position B3 is Lys and position B29 is Glu or Asp.

The acylated, single-chain insulin according to the invention and the rapid acting human insulin or human insulin analogue can be mixed in a ratio from about 90/10%; about 70/30% or about 50/50%.

Antidiabetic agents will include insulin, GLP-1 (1-37) (glucagon like peptide-1) described in WO 98/08871, WO 99/43706, U.S. Pat. No. 5,424,286 and WO 00/09666, GLP-2, exendin-4(1-39), insulinotropic fragments thereof, insulinotropic analogues thereof and insulinotropic derivatives thereof. Insulinotropic fragments of GLP-1 (1-37) are insulinotropic peptides for which the entire sequence can be found in the sequence of GLP-1 (1-37) and where at least one terminal amino acid has been deleted.

### DESCRIPTION OF THE INVENTION

The present acylated, single chain insulin analogues are acylated in a certain area in the A-chain which is situated on the surface of the insulin molecule and will not interfere with the hexamer formation of the single-chain insulin molecules.

The single-chain insulin is acylated by well known technology and the acyl group will typically be derived from a mono- or dicarboxylic fatty acid which may be linear or branched and which has at least 2 carbon atoms.

The acyl group may be a lipophilic group and may be a monocarboxylic or dicarboxylic fatty acid moiety having from about 6 to about 32 carbon atoms which may comprise at least one free carboxylic acid group or a group which is negatively charged at neutral pH.

The fatty acid may furthermore be saturated or unsaturated and may comprise one or more heteroatoms like O and S and one or more heterocyclic ring systems.

Non limiting examples of monocarboxylic fatty acids are capric acid, lauric acid, tetradecanoic acid (myristic acid), pentadecanoic acid, palmitic acid, heptadecanoic acid, stearic acid, dodecanoic acid, tridecanoic acid, and tetradecanoic acid.

Non limiting examples of dicarboxylic fatty are succinic acid, hexanedioic acid, octanedioic acid, decanedioic acid, dodecanedioic acid, tetradecanedioic acid, hexadecanedioic acid, heptadecanedioic acid, and octadecanedioic acid.

The fatty acid moiety will more typically have from 6 to 24, from 8 to 20, from 12 to 20, from 12-16, from 10-16, from 10-20, from 14-18 or from 14-16 carbon atoms.

The acyl group may also be a lipophilic substituent selected from the group comprising CH₃(CH₂)ₙCO—, wherein n is 4 to 24, such as CH₃(CH₂)₆CO—, CH₃(CH₂)₈CO—, CH₃(CH₂)₁₀CO—, CH₃(CH₂)₁₂CO—, CH₃(CH₂)₁₄CO—, CH₃(CH₂)₁₆CO—, CH₃(CH₂)₁₈CO—, CH₃(CH₂)₂₀CO— and CH₃(CH₂)₂₂CO—.

In one embodiment the acyl group is a straight-chain or branched α,ω-dicarboxylic acid. In another embodiment the acyl group may have the formula HOOC(CH₂)ₘCO—, wherein m is 2 to 24, such as HOOC(CH₂)₁₄CO—, HOOC(CH₂)₁₆CO—, HOOC(CH₂)₁₈CO—, HOOC(CH₂)₂₀CO— and HOOC(CH₂)₂₂CO—.

Finally, the acyl group may by a lithocholic acid.

The acyl group may be attached to the single-chain insulin by a linker molecule, e.g. a suitable amino acid residue. In one aspect the linker comprises 1-4 amino acid residues linked together via amide bonds of which one may comprise a free carboxylic acid group or a group which is negatively charged at neutral pH.

In another aspect the linker is an amino acid residue, a peptide chain of 2-4 amino acid residues or is α-Asp; β-Asp; α-Glu; γ-Glu; α-hGlu; δ-hGlu; —N(CH₂COOH)CH₂CO—; —N(CH₂CH₂COOH)CH₂CH₂CO—; —N(CH₂COOH)CH₂CH₂CO— or —N(CH₂CH₂COOH)CH₂CO—.

In a further aspect the linker can be a chain composed of two amino acid residues of which one has from 4 to 10 carbon atoms and a carboxylic acid group in the side chain while the other has from 2 to 11 carbon atoms but no free carboxylic acid group. The amino acid residue with no free carboxylic acid group can be a neutral α-amino acid residue. Examples of such linkers are: α-Asp-Gly; Gly-α-Asp; β-Asp-Gly; Gly-β-Asp; α-Glu-Gly; Gly-α-Glu; γ-Glu-Gly; Gly-γ-Glu; α-hGlu-Gly; Gly-α-hGlu; δ-hGlu-Gly; and Gly-δ-hGlu.

In a further aspect the linker is a chain composed of two amino acid residues, independently having from 4 to 10 carbon atoms, and both having a carboxylic acid group in the side chain. One of these amino acid residues or both of them can be α-amino acid residues. Examples of such linkers are: α-Asp-α-Asp; α-Asp-α-Glu; α-Asp-α-hGlu; α-Asp-β-Asp; α-Asp-γ-Glu; α-Asp-α-hGlu; β-Asp-α-Asp; β-Asp-α-Glu; β-Asp-α-hGlu; β-Asp-β-Asp; β-Asp-γ-Glu; β-Asp-δ-hGlu; α-Glu-α-Asp; α-Glu-α-Glu; α-Glu-α-hGlu; α-Glu-β-Asp; α-Glu-γ-Glu; α-Glu-δ-hGlu; γ-Glu-α-Asp; γ-Glu-α-Glu; γ-Glu-α-hGlu; γ-Glu-β-Asp; γ-Glu-γ-Glu; γ-Glu-δ-hGlu; α-hGlu-α-Asp; α-hGlu-α-Glu; α-hGlu-α-hGlu; α-hGlu-β-Asp; α-hGlu-γ-Glu; α-hGlu-δ-hGlu; δ-hGlu-α-Asp; δ-hGlu-α-Glu; δ-hGlu-α-hGlu; δ-hGlu-β-Asp; δ-hGlu-γ-Glu; and δ-hGlu-δ-hGlu.

In a further aspect the linker is a chain composed of three amino acid residues, independently having from 4 to 10 carbon atoms, the amino acid residues of the chain being selected from the group of residues having a neutral side chain and residues having a carboxylic acid group in the side chain so that the chain has at least one residue which has a carboxylic acid group in the side chain. In one aspect, the amino acid residues are α-amino acid residues.

In a further aspect, the linker is a chain composed of four amino acid residues, independently having from 4 to 10 carbon atoms, the amino acid residues of the chain being selected from the group having a neutral side chain and residues having a carboxylic acid group in the side chain so that the chain has at least one residue which has a carboxylic acid group in the side chain. In one aspect, the amino acid residues are α-amino acid residues.

The linker may comprise one or more aromatic ring systems which may be substituted with a carboxylic acid or a carboxy amide group.

The linker and the acyl group may thus have the formula CH₃(CH₂)ₙCONH—CH(COOH)—(CH₂)ₚCO—, wherein n is an integer of from 4-24, 10-24 or 8-24 and p is an integer of from 1-3. In another embodiment the linker and the acyl group have the formula HOOC(CH₂)ₙCONH—CH(COOH)—(CH₂)ₚCO—, wherein n is an integer of from 4-24 and p is an integer of from 1-3. In another embodiment the combination of the linker and the acyl group has the formula CH₃(CH₂)ₙCONH—CH(CH₂)ₚ(COOH)CO— wherein n is an integer of from 4-24 and p is an integer of from 1-3 or HOOC(CH₂)ₙCONH—CH((CH₂)ₚCOOH)CO—, wherein n is an integer of from 4-24 and p is an integer of from 1-3.

Acyl groups and linkers suitable for use in the present invention are disclosed in WO 95/07931, WO 96/00107, WO 97/31022, WO 2005/012347 and EP 894095.

Acylation of the single-chain insulins according to the present invention can be made by a methods analogue to the methods disclosed in U.S. Pat. Nos. 5,750,497 and 5,905,140. Methods for acylation are further described in the experimental part.

As mentioned above the connecting peptide may vary in length and in the composition of the amino acid sequence. Non limiting examples of connecting peptides suitable for the present invention are disclosed in WO 2005/054291.

Non limiting examples of acylated, single-chain insulins according to the present invention are

B(1-29)-B29A-VGLSSGQ-A(1-21)-A18K(N(eps)myristoyl) human insulin;
B(1-29)-B29A-VGLSSGQ-A(1-21)-A18K(N(eps)octadecandioyl) human insulin;
B(1-29)-B29A-VGLSSGQ-A(1-21)-A18K(N(eps)hexadecandioyl-γ-L-Glu) human insulin;
B(1-29)-B29A-VGLSSGQ-A(1-21)-A21K(N(eps)myristoyl) human insulin;
B(1-29)-B29A-VGLSSGQ-A(1-21)-A21K(N(eps)octadecandioyl) human insulin;
B(1-29)-B29A-VGLSSGQ-A(1-21)-A21K(N(eps)hexadecandioyl-γ-L-Glu) human insulin;
B(1-29)-B29A-VGLSSGQ-A(1-21)-A22K(N(eps)myristoyl) human insulin;
B(1-29)-B29A-VGLSSGQ-A(1-21)-A22K(N(eps)octadecandioyl) human insulin;
B(1-29)-B29A-VGLSSGQ-A(1-21)-A22K(N(eps)hexadecandioyl-γ-L-Glu) human insulin;
B(1-29)-B29A-VGLSSGQ-A(1-21)-A22G-A23K(N(eps)myristoyl) human insulin;
B(1-29)-B29A-VGLSSGQ-A(1-21)-A22G-A23K(N(eps)octadecandioyl) human insulin;
B(1-29)-B29A-VGLSSGQ-A(1-21)-A22G-A23K(N(eps)hexadecandioyl-γ-L-Glu) human insulin;
B(1-29)-B29A-TGLGSGR-A(1-21)-A18K(N(eps)myristoyl) human insulin;
B(1-29)-B29A-TGLGSGR-A(1-21)-A18K(N(eps)octadecandioyl) human insulin;
B(1-29)-B29A-TGLGSGR-A(1-21)-A18K(N(eps)hexadecandioyl-γ-L-Glu) human insulin;
B(1-29)-B29A-TGLGSGR-A(1-21)-A21K(N(eps)myristoyl) human insulin;
B(1-29)-B29A-TGLGSGR-A(1-21)-A21K(N(eps)octadecandioyl) human insulin;
B(1-29)-B29A-TGLGSGR-A(1-21)-A21K(N(eps)hexadecandioyl-γ-L-Glu) human insulin;
B(1-29)-B29A-TGLGSGR-A(1-21)-A22K(N(eps)myristoyl) human insulin;
B(1-29)-B29A-TGLGSGR-A(1-21)-A22K(N(eps)octadecandioyl) human insulin;
B(1-29)-B29A-TGLGSGR-A(1-21)-A22K(N(eps)hexadecandioyl-γ-L-Glu) human insulin;
B(1-29)-B29A-TGLGSGR-A(1-21)-A22G-A23K(N(eps)myristoyl) human insulin;
B(1-29)-B29A-TGLGSGR-A(1-21)-A22G-A23K(N(eps)octadecandioyl) human insulin;
B(1-29)-B29A-TGLGSGR-A(1-21)-A22G-A23K(N(eps)hexadecandioyl-γ-L-Glu) human insulin;
B(1-29)-B29H-VGLSSGQ-A(1-21)-A18K(N(eps)myristoyl) human insulin;
B(1-29)-B29H-VGLSSGQ-A(1-21)-A18K(N(eps)octadecandioyl) human insulin;
B(1-29)-B29H-VGLSSGQ-A(1-21)-A18K(N(eps)hexadecandioyl-γ-L-Glu) human insulin;
B(1-29)-B29H-VGLSSGQ-A(1-21)-A21K(N(eps)myristoyl) human insulin;
B(1-29)-B29H-VGLSSGQ-A(1-21)-A21K(N(eps)octadecandioyl) human insulin;
B(1-29)-B29H-VGLSSGQ-A(1-21)-A21K(N(eps)hexadecandioyl-γ-L-Glu) human insulin;
B(1-29)-B29H-VGLSSGQ-A(1-21)-A22K(N(eps)myristoyl) human insulin;
B(1-29)-B29H-VGLSSGQ-A(1-21)-A22K(N(eps)octadecandioyl) human insulin;
B(1-29)-B29H-VGLSSGQ-A(1-21)-A22K(N(eps)hexadecandioyl-γ-L-Glu) human insulin;
B(1-29)-B29H-VGLSSGQ-A(1-21)-A22G-A23K(N(eps)myristoyl) human insulin;
B(1-29)-B29H-VGLSSGQ-A(1-21)-A22G-A23K(N(eps)octadecandioyl) human insulin;
B(1-29)-B29H-VGLSSGQ-A(1-21)-A22G-A23K(N(eps)hexadecandioyl-γ-L-Glu) human insulin;
B(1-29)-B29H-TGLGSGR-A(1-21)-A18K(N(eps)myristoyl) human insulin;
B(1-29)-B29H-TGLGSGR-A(1-21)-A18K(N(eps)octadecandioyl) human insulin;
B(1-29)-B29H-TGLGSGR-A(1-21)-A18K(N(eps)hexadecandioyl-γ-L-Glu) human insulin;
B(1-29)-B29H-TGLGSGR-A(1-21)-A21K(N(eps)myristoyl) human insulin;
B(1-29)-B29H-TGLGSGR-A(1-21)-A21K(N(eps)octadecandioyl) human insulin;
B(1-29)-B29H-TGLGSGR-A(1-21)-A21K(N(eps)hexadecandioyl-γ-L-Glu) human insulin;
B(1-29)-B29H-TGLGSGR-A(1-21)-A22K(N(eps)myristoyl) human insulin;
B(1-29)-B29H-TGLGSGR-A(1-21)-A22K(N(eps)octadecandioyl) human insulin;
B(1-29)-B29H-TGLGSGR-A(1-21)-A22K(N(eps)hexadecandioyl-γ-L-Glu) human insulin;
B(1-29)-B29Q-VGLSSGQ-A(1-21)-A18K(N(eps)myristoyl)-human insulin;
B(1-29)-B29Q-VGLSSGQ-A(1-21)-A18K(N(eps)octadecandioyl)-human insulin;
B(1-29)-B29Q-VGLSSGQ-A(1-21)-A18K(N(eps)hexadecandioyl-γ-L-Glu)-human insulin;
B(1-29)-B29Q-VGLSSGQ-A(1-21)-A21K(N(eps)myristoyl) human insulin;
B(1-29)-B29Q-VGLSSGQ-A(1-21)-A21K(N(eps)octadecandioyl) human insulin;
B(1-29)-B29Q-VGLSSGQ-A(1-21)-A21K(N(eps)hexadecandioyl-γ-L-Glu) human insulin;
B(1-29)-B29Q-VGLSSGQ-A(1-21)-A22K(N(eps)myristoyl) human insulin;
B(1-29)-B29Q-VGLSSGQ-A(1-21)-A22K(N(eps)octadecandioyl) human insulin;
B(1-29)-B29Q-VGLSSGQ-A(1-21)-A22K(N(eps)hexadecandioyl-γ-L-Glu) human insulin;
B(1-29)-B29Q-TGLGSGR-A(1-21)-A18K(N(eps)myristoyl) human insulin;
B(1-29)-B29Q-TGLGSGR-A(1-21)-A18K(N(eps)octadecandioyl) human insulin;
B(1-29)-B29Q-TGLGSGR-A(1-21)-A18K(N(eps)hexadecandioyl-γ-L-Glu) human insulin;
B(1-29)-B29Q-TGLGSGR-A(1-21)-A21K(N(eps)myristoyl) human insulin;
B(1-29)-B29Q-TGLGSGR-A(1-21)-A21K(N(eps)octadecandioyl) human insulin;
B(1-29)-B29Q-TGLGSGR-A(1-21)-A21K(N(eps)hexadecandioyl-γ-L-Glu) human insulin;
B(1-29)-B29Q-TGLGSGR-A(1-21)-A22K(N(eps)myristoyl) human insulin;
B(1-29)-B29Q-TGLGSGR-A(1-21)-A22K(N(eps)octadecandioyl) human insulin; and
B(1-29)-B29Q-TGLGSGR-A(1-21)-A22K(N(eps)hexadecandioyl-γ-L-Glu) human insulin.

The parent single-chain insulins are produced by expressing a DNA sequence encoding the single-chain insulin in question in a suitable host cell by well known technique as disclosed in e.g. EP patent 1692168 or U.S. Pat. No. 6,500,645. The single-chain insulin is either expressed directly or as a precursor molecule which has an N-terminal extension on the B-chain. This N-terminal extension may have the function of increasing the yield of the directly expressed product and may be of up to 15 amino acid residues long. The N-terminal extension is to be cleaved of in vitro after isolation from the culture broth and will therefore have a cleavage site next to B1. N-terminal extensions of the type suitable in the present invention are disclosed in U.S. Pat. No. 5,395,922, and European Patent No. 765,395A.

The isolated insulin precursor can be acylated in the desired position as well know with the art and examples of such insulin analogues are described e.g. in the European patent applications having the publication Nos. EP 214826, EP 375437 and EP 383472.

The polynucleotide sequence coding for the respective insulin polypeptide may be pre-pared synthetically by established standard methods, e.g. the phosphoamidite method described by Beaucage et al. (1981) Tetrahedron Letters 22:1859-1869, or the method described by Matthes et al. (1984) EMBO Journal 3:801-805. A currently preferred way of pre-paring the DNA construct is by polymerase chain reaction (PCR).

The polynucleotide sequences may also be of mixed genomic, cDNA, and synthetic origin. For example, a genomic or cDNA sequence encoding a leader peptide may be joined to a genomic or cDNA sequence encoding the A and B chains, after which the DNA sequence may be modified at a site by inserting synthetic oligonucleotides encoding the desired amino acid sequence for homologous recombination in accordance with well-known procedures or preferably generating the desired sequence by PCR using suitable oligonucleotides.

The recombinant vector capable of replicating in the selected microorganism or host cell and which carries a polynucleotide sequence encoding the insulin polypeptide in question may be an autonomously replicating vector e.g., a plasmid, an extra-chromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. The vector may be linear or closed circular plasmid.

In one embodiment, the recombinant expression vector is capable of replicating in yeast. Examples of sequences which enable the vector to replicate in yeast are the yeast plasmid 2 μm replication genes REP 1-3 and origin of replication.

The vectors may contain one or more selectable markers which permit easy selection of transformed cells. Examples of bacterial selectable markers are the dal genes from Bacillus subtilis or Bacillus licheniformis, or markers which confer antibiotic resistance such as ampicillin, kanamycin, chloramphenicol or tetracycline resistance. Suitable markers for yeast host cells are ADE2, HIS3, LEU2, LYS2, MET3, TRP1, and URA3. A well suited selectable marker for yeast is the Schizosaccharomyces pompe TPI gene (Russell (1985) Gene 40:125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intra-cellular polypeptides either homologous or heterologous to the host cell. In a yeast host, useful promoters are the Saccharomyces cerevisiae Ma1, TPI, ADH or PGK promoters.

The polynucleotide construct will also typically be operably connected to a suitable terminator. In yeast a suitable terminator is the TPI terminator (Alber et al. (1982) J. Mol. Appl. Genet. 1:419-434).

The vector comprising such polynucleotide sequence is introduced into the host cell so that the vector is maintained as a chromosomal integrant or as a self-replicating extrachromosomal vector as described earlier. The term “host cell” encompasses any progeny of a parent cell that is not identical to the parent cell due to mutations that occur during replication. The host cell is typically a yeast cell. The yeast organism used in the process of the invention may be any suitable yeast organism which, on cultivation, produces large amounts of the single chain insulin of the invention. Examples of suitable yeast organisms are strains selected from the yeast species Saccharomyces cerevisiae, Saccharomyces kluyveri, Schizosaccharomyces pombe, Sacchoromyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans.

The transformation of the yeast cells may for instance be effected by protoplast formation followed by transformation in a manner known per se. The medium used to cultivate the cells may be any conventional medium suitable for growing yeast organisms. The secreted insulin polypeptide, a significant proportion of which will be present in the medium in correctly processed form, may be recovered from the medium by conventional procedures including separating the yeast cells from the medium by centrifugation, filtration or catching the insulin precursor by an ion exchange matrix or by a reverse phase absorption matrix, precipitating the proteinaceous components of the supernatant or filtrate by means of a salt, e.g. ammonium sulphate, followed by purification by a variety of chromatographic procedures, e.g. ion exchange chromatography, affinity chromatography, or the like.

### Pharmaceutical Compositions

Pharmaceutical compositions containing the acylated, single-insulins of this invention can be used in the treatment of states which are sensitive to insulin. Thus, they can be used in the treatment of type 1 diabetes, type 2 diabetes and hyperglycaemia for example as sometimes seen in seriously injured persons and persons who have undergone major surgery. The optimal dose level for any patient will depend on a variety of factors including the efficacy of the specific insulin derivative employed, the age, body weight, physical activity, and diet of the patient, on a possible combination with other drugs, and on the severity of the state to be treated. It is recommended that the daily dosage of the acylated, single-chain insulin of this invention be determined for each individual patient by those skilled in the art in a similar way as for known insulin compositions.

Pharmaceutical compositions containing an acylated, single-chain insulins according to the present invention may be administered parenterally to patients in need of such a treatment. Parenteral administration may be performed by subcutaneous, intramuscular or intravenous injection by means of a syringe. Usually, the pharmaceutical compositions will be administered subcutaneously. However the acylated, single-chain insulins of the invention may also be formulated for pulmunal administration.

In another embodiment the pharmaceutical formulations may be used in connection with pen-like injection devices, which may be prefilled and disposable, or the insulin preparations may be supplied from a reservoir which is removable. Non-limiting examples of pen-like injection devices are FlexPen®, InnoLet®, InDuo™, Innovo®.

The acylated single-chain insulins of this invention may be delivered by a dry powder inhaler or a sprayer. A device suitable for pulmonary administration of aqueous insulin preparations is the AerX® device.

Other examples of commercially available inhalation devices suitable for administration are Turbohaler™ (Astra), Rotahaler® (Glaxo), Diskus® (Glaxo), Spiros™ inhaler (Dura), devices marketed by Inhale Therapeutics, the Ultravent® nebulizer (Mallinckrodt), the Acorn II® nebulizer (Marquest Medical Products), the Ventolin® metered dose inhaler (Glaxo), the Spinhaler® powder inhaler (Fisons), or the like.

Injectable compositions of the acylated, single-chain insulins of the invention can be prepared using the conventional techniques of the pharmaceutical industry which involve dissolving and mixing the ingredients as appropriate to give the desired end product. Thus, according to one procedure, an acylated, single-chain insulin according to the invention is dissolved in an amount of water which is somewhat less than the final volume of the composition to be prepared. An isotonic agent, a preservative and a buffer is added as required and the pH value of the solution is adjusted—if necessary—using an acid, e.g. hydrochloric acid, or a base, e.g. aqueous sodium hydroxide as needed. Finally, the volume of the solution is adjusted with water to give the desired concentration of the ingredients.

Pharmaceutical compositions of the claimed acylated, single-chain insulins will contain usual adjuvants and additives and are preferably formulated as an aqueous solution. The aqueous medium is made isotonic, for example, with sodium chloride, sodium acetate or glycerol. Furthermore, the aqueous medium may contain zinc ions, buffers and preservatives. The pH value of the composition is adjusted to the desired value and may be between about 4 to about 8.5, preferably between 7 and 7.5 depending on the isoelectric point, pl, of the single-chain insulin in question.

Consequently, this invention also relates to a pharmaceutical composition containing an acylated, single-chain insulin of the invention and optionally one or more agents suitable for stabilization, preservation or isotonicity, for example, zinc ions, phenol, cresol, a parabene, sodium chloride, glycerol or mannitol. The zinc content of the present formulations may be between 0 and about 6 zinc atoms per insulin hexamer. The acylated, single-chain insulins may also be formulated with IFD ligands as disclosed in WO 2003027081.

The buffer used in the pharmaceutical preparation according to the present invention may be selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof.

The pharmaceutically acceptable preservative may be selected from the group consisting of phenol, o-cresol, m-cresol, p-cresol, methyl p-hydroxybenzoate, propyl phydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19th edition, 1995.

The isotonicity agent may be selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or watersoluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19th edition, 1995.

The pharmaceutical composition may be a solution containing from about 120 nmol/ml to about 2400 nmol/ml, from about 400 nmol/ml to about 2400 nmol/ml, from about 400 nmol/ml to about 1200 nmol/ml, from about 600 nmol/ml to about 2400 nmol/ml, or from about 600 nmol/ml to about 1200 nmol/ml of the acylated, single-chain insulin according to the invention or of a mixture of the acylated, single-chain insulin according to the invention with a fast acting insulin analogue.

Whenever an acylated, single chain insulin of this invention is combined with another form of treatment, this administration can be simultaneous or sequential, in a manner effective to result in their combined actions within the subject treated.

In one embodiment, the acylated, single-chain insulin may be administered in combination with human insulin or a fast acting analogue of human insulin as described above, either as a pre-mixed preparation, or by substantially simultaneous administration of two separate preparations, or by sequential administration, i.e. administrations may be separated in time. The agents would be provided in amounts effective and for periods of time effective to result in their combined presence and their combined actions.

The acylated single-chain insulins according to the present invention may also be used on combination treatment together with an oral antidiabetic such as a thiazolidindione, metformin and other type 2 diabetic pharmaceutical preparation for oral treatment.

Furthermore, the acylated, single-chain insulin according to the invention may be administered in combination with one or more antiobesity agents or appetite regulating agents.

Such agents may be selected from the group consisting of CART (cocaine amphetamine regulated transcript) agonists, NPY (neuropeptide Y) antagonists, MC3 (melanocortin 3) agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, 3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti related protein) inhibitors, H3 histamine antagonists such as those disclosed in WO 00/42023, WO 00/63208 and WO 00/64884, which are incorporated herein by reference, exendin-4, GLP-1 agonists, ciliary neurotrophic factor, and oxyntomodulin. Further antiobesity agents are bupropion (antidepressant), topiramate (anticonvulsant), ecopipam (dopamine D1/D5 antagonist) and naltrexone (opioid antagonist).

In one embodiment the antiobesity agent is leptin, a serotonin and norepinephrine reuptake inhibitor eg sibutramine, a lipase inhibitor eg orlistat, an adrenergic CNS stimulating agent eg dexamphetamine, amphetamine, phentermine, mazindol phendimetrazine, diethylpropion, fenfluramine or dexfenfluramine.

It should be understood that any suitable combination of the acylated, single-chain insulins with diet and/or exercise, one or more of the above-mentioned compounds and optionally one or more other active substances are considered to be within the scope of the present invention.

With Insulin as used herein is meant human insulin with disulfide bridges between Cys^{A7} and Cys^{B7} and between Cys^{A20} and Cys^{B19} and an internal disulfide bridge between Cys^{A6} and Cys^{A11}, porcine insulin and bovine insulin.

By “insulin analogue” as used herein is meant a polypeptide which has a molecular structure which formally can be derived from the structure of a naturally occurring insulin, for example that of human insulin, by deleting and/or substituting at least one amino acid residue occurring in the natural insulin and/or by adding at least one amino acid residue. The added and/or substituted amino acid residues can either be codable amino acid residues or other naturally occurring amino acid residues or purely synthetic amino acid residues.

By a single-chain insulin is meant a polypeptide sequence of the general structure B-C-A wherein B is the human B insulin chain or an analogue or derivative thereof, A is the human insulin A chain or an analogue or derivative and C is a peptide chain connecting the C-terminal amino acid residue in the B-chain (normally B30) with A1. If the B chain is a desB30 chain the connecting peptide will connect B29 with A1. The single-chain insulin will contain correctly positioned disulphide bridges (three) as in human insulin that is between CysA7 and CysB7 and between CysA20 and CysB19 and an internal disulfide bridge between CysA6 and CysA11.

Analogues of the B-chain may be such wherein the amino acid residue in B1 is substituted with another amino acid residue such as Asp or Gly or is deleted. Also Asn at position B3 may be mutated with Thr, Gln, Glu or Asp. The B-chain may also comprise an N-terminal extension or the B30 amino acid residue may be deleted.

Analogues of the A chain may be such wherein the amino acid residue in position A18 is substituted with another amino acid residue, such as Gln. Also, Asn at position A21 may be mutated with Ala, Gln, Glu, Gly, H is, Ile, Leu, Met, Ser, Thr, Trp, Tyr or Val, in particular with Gly, Ala, Ser, or Thr and preferably with Gly. The A chain may de extended at its C-terminal end by one or two amino acid residues which are denoted A22 and A23, respectively. Either A22 or A23 may be acylated according to the present invention. When A23 is acylated then the amino acid in position A22 may by any amino acid residue except Cys and Lys.

With desB30 or B(1-29) is meant a natural insulin B chain or an analogue thereof lacking the B30 amino acid residue, A(1-21) means the natural insulin A chain or an analogue or derivative thereof. The amino acid residues are indicated in the three letter amino acid code or the one letter amino code.

With B1, A1 etc. is meant the amino acid residue in position 1 in the B chain of insulin (counted from the N-terminal end) and the amino acid residue in position 1 in the A chain of insulin (counted from the N-terminal end), respectively.

The single-chain insulins are named according to the following rule: The sequence starts with the B-chain, continues with the C-peptide and ends with the A-chain. The amino acid residues are named after their respective counterparts in human insulin and mutations and acylations are explicitly described whereas unaltered amino acid residues in the A- and B-chains are not mentioned. For example, an insulin having the following mutations as compared to human insulin: A21G,A8Lys,A18Q, desB30 and the connecting TGLGSGR (SEQ ID NO:2) connecting the C-terminal B-chain and the N-terminal A-chain is named B(1-29)TGLGSGR-A(1-21)-A8Lys,A18Q,A21G human insulin.

By acylation is understood the chemical reaction whereby a hydrogen of an amino group or hydroxy group is exchanged with an acyl group.

With fatty acid is meant a linear or branched carboxylic acid having at least 2 carbon atoms and being saturated or unsaturated.

With fatty diacid is meant a linear or branched dicarboxylic acid having at least 2 carbon atoms and being saturated or unsaturated.

With fast acting insulin is meant an insulin having a faster onset of action than normal or regular human insulin.

With long acting insulin is meant an insulin having a longer duration of action than normal or regular human insulin.

With connecting peptide is meant a peptide chain which connects the C-terminal amino acid residue of the B-chain with the N-terminal amino acid residue of the A-chain.

The term basal insulin as used herein means an insulin peptide which has a time-action of more than 8 hours, in particularly of at least 9 hours. Preferably, the basal insulin has a time-action of at least 10 hours. The basal insulin may thus have a time-action in the range from 9 to 15 hours.

With parent insulin is meant the single-chain insulin peptide back bone chain with the modifications in the amino acid residue composition according to the present invention.

By single-chain insulin having insulin activity is meant single-chain insulin with the ability to lower the blood glucose in mammalians as measured in a suitable animal model, which may be a rat, rabbit, or pig model, after suitable administration e.g. by intravenous or subcutaneous administration.

By soluble at neutral pH is meant that a 0.6 mM single chain insulin is soluble at neutral pH.

By high physical stability is meant a tendency to fibrillation being less than 50% of that of human insulin. Fibrillation may be described by the lag time before fibril formation is initiated at a given conditions.

With the term lipophilic is meant the product in question can dissolve in lipids.

By fibrillation is meant a physical process by which partially unfolded insulin molecules interacts with each other to form linear aggregates.

A polypeptide with Insulin receptor and IGF-1 receptor affinity is a polypeptide which is capable of interacting with an insulin receptor and a human IGF-1 receptor in a suitable binding assay. Such receptor assays are well-know within the field.

The term analogue as used herein referring to a peptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. In one embodiment an analogue comprises less than 5 modifications (substitutions, deletions, additions) relative to the native peptide. In another embodiment an analogue comprises less than 4 modifications (substitutions, deletions, additions) relative to the native peptide. In another embodiment an analogue comprises less than 3 modifications (substitutions, deletions, additions) relative to the native peptide. In another embodiment an analogue comprises less than 2 modifications (substitutions, deletions, additions) relative to the native peptide. In another embodiment an analogue comprises only a single modification (substitutions, deletions, additions) relative to the native peptide.

In the present context, the unit “U” corresponds to 6 nmol.

The term effective amount as used herein means a dosage which is sufficient in order for the treatment of the patient to be effective compared with no treatment.

POT″ is the Schizosaccharomyces pombe triose phosphate isomerase gene, and “TPI1” is the S. cerevisiae triose phosphate isomerase gene.

The term “signal peptide” is understood to mean a pre-peptide which is present as an N-terminal sequence on the precursor form of a protein. The function of the signal peptide is to allow the heterologous protein to facilitate translocation into the endoplasmic reticulum. The signal peptide is normally cleaved off in the course of this process. The signal peptide may be heterologous or homologous to the yeast organism producing the protein. A number of signal peptides which may be used with the DNA construct of the invention including yeast aspartic protease 3 (YAP3) signal peptide or any functional analog (Egel-Mitani et al. (1990) YEAST 6:127-137 and U.S. Pat. No. 5,726,038) and the α-factor signal of the MFα1 gene (Thorner (1981) in The Molecular Biology of the Yeast Saccharomyces cerevisiae, Strathern et al., eds., pp 143-180, Cold Spring Harbor Laboratory, NY and U.S. Pat. No. 4,870,00.

The term “pro-peptide” means a polypeptide sequence whose function is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The pro-peptide may be the yeast α-factor pro-peptide, vide U.S. Pat. Nos. 4,546,082 and 4,870,008. Alternatively, the pro-peptide may be a synthetic pro-peptide, which is to say a pro-peptide not found in nature. Suitable synthetic pro-peptides are those disclosed in U.S. Pat. Nos. 5,395,922; 5,795,746; 5,162,498 and WO 98/32867. The pro-peptide will preferably contain an endopeptidase processing site at the C-terminal end, such as a LysArg sequence or any functional analogue thereof.

In the present context the three-letter or one-letter indications of the amino acids have been used in their conventional meaning as indicated in the following. Unless indicated explicitly, the amino acids mentioned herein are L-amino acids. Further, the left and right ends of an amino acid sequence of a peptide are, respectively, the N- and C-termini unless otherwise specified.

Abbreviations for amino acids

| Amino acid | Three-letter code | One-letter code |
|---|---|---|
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The following abbreviations have been used in the specification and examples:

Bzl=Bn: benzyl

DIEA: N,N-diisopropylethylamine

DMF: N,N-dimethylformamide

tBu: tert-butyl

Glu: Glutamic acid

TSTU: O-(N-succinimidyl)-1,1,3,3-tetramethyluronium tetrafluoroborate

THF: Tetrahydrofuran

EtOAc: Ethyl acetate

DIPEA: Diisopropylethylamine

HOAt: 1-Hydroxy-7-azabenzotriazole

NMP: N-methylpyrrolidin-2-one

TEA: triethyl amine

Su: succinimidyl=2,5-dioxo-pyrrolidin-1-yl

TFA: trifluoracetic acid

DCM: dichloromethane

DMSO: dimethyl sulphoxide

RT: room temperature

cyano: a/pha-cyano-4-hydroxycinnamic acid

The present invention is described in further detail in the following examples which are not in any way intended to limit the scope of the invention as claimed. All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

### EXAMPLES

### General Methods

### Acylation

The following examples and general procedures refer to intermediate compounds and final products identified in the specification and in the synthesis schemes. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

The compounds of the invention can be purified by employing one or more of the following procedures which are typical within the art. These procedures can—if needed—be modified with regard to gradients, pH, salts, concentrations, flow, columns and so forth. Depending on factors such as impurity profile, solubility of the insulins in question etcetera, these modifications can readily be recognised and made by a person skilled in the art.

After acidic HPLC or desalting, the compounds are isolated by lyophilisation of the pure fractions.

After neutral HPLC or anion exchange chromatography, the compounds are desalted, precipitated at isoelectrical pH, or purified by acidic HPLC.

### Typical Purification Procedures:

The HPLC system is a Gilson system consisting of the following: Model 215 Liquid handler, Model 322-H2 Pump and a Model 155 UV Dector. Detection is typically at 210 nm and 280 nm.

The Äkta Purifier FPLC system (Amersham Biosciences) consists of the following:

Model P-900 Pump, Model UV-900 UV detector, Model pH/C-900 pH and conductivity detector, Model Frac-950 Fraction collector. UV detection is typically at 214 nm, 254 nm and 276 nm.

Acidic HPLC:

| Column: | Macherey-Nagel SP 250/21 Nucleusil 300-7 C4 | |
|---|---|---|
| Flow: | 8 ml/min, | |
| Buffer A: | 0.1% TFA in acetonitrile | |
| Buffer B: | 0.1% TFA in water. | |
| Gradient: | 0.0-5.0 min: | 10% A |
| | 5.00-30.0 min: | 10% A to 90% A |
| | 30.0-35.0 min: | 90% A |
| | 35.0-40.0 min: | 100% A |

Neutral HPLC:

| Column: | Phenomenex, Jupiter, C4 5 μm 250 × 10.00 mm, 300 Å | |
|---|---|---|
| Flow: | 6 ml/min | |
| Buffer A: | 5 mM TRIS, 7.5 mM (NH₄)₂SO₄, pH = 7.3, 20% CH₃CN | |
| Buffer B: | 60% CH₃CN, 40% water | |
| Gradient: | 0-5 min: | 10% B, |
| | 5-35 min: | 10-60% B |
| | 35-39 min: | 60% B, |
| | 39-40 min: | 70% B |
| | 40-43.5 min: | 70% B |

Anion exchange chromatography:

| | |
|---|---|
| Column: | RessourceQ, 6 ml |
| Flow: | 6 ml/min |
| Buffer A: | 0.09% NH₄HCO₃, 0.25% NH₄OAc, 42.5% ethanol pH 8.4 |
| Buffer B: | 0.09% NH₄HCO₃, 2.5% NH₄OAc, 42.5% ethanol pH 8.4 |
| Gradient: | 100% A to 100% B during 30 column volumes |

Desalting:

| | |
|---|---|
| Column: | HiPrep 26/10 |
| Flow: | 10 ml/min, 6 column volumes |
| Buffer: | 10 mM NH₄HCO₃ |

### Analytical Procedures:

Method 1:
Two Waters 510 HPLC pumps
Waters 2487 Dual λ Absorbance detector

| | |
|---|---|
| Buffer A: | 0.1% TFA in acetonitrile. |
| Buffer B: | 0.1% TFA in water. |
| Flow: | 1.5 ml/min. |
| Gradient: | 1-17 min: 25% B to 85% B, 17-22 min: 85% B, 22-23 min: |
| | 85% B to 25% B, 23-30 min 25% B, 30-31 min 25% B flow: |
| | 0.15 ml/min. |
| Column: | C4 5μ 150 × 4_60 mm “phenomenex, Jupiter”. |
| Detection: | UV 214 nm. |

Method 2:
Two Waters 510 HPLC pumps
Waters 2487 Dual λ Absorbance detector

| | |
|---|---|
| BufferA: | 0.1% TFA, 10% CH₃CN, 89.9% water. |
| Buffer B: | 0.1% TFA, 80% CH₃CN, 19.9% water. |
| Flow: | 1.5 ml/min. |
| Gradient: | 0-17 min: 20%-90% B, 17-21 min 90% B. |
| Column: | C4 5μ 150 × 4_60 mm “phenomenex, Jupiter”, kept at 40° C. |
| Detection: | UV 214 nm. |

Method 3:
Two Waters 510 HPLC pumps
Waters 486 Tunable Absorbance Detector
Waters 717 Autosampler

| | |
|---|---|
| Column: | C4 5μ 150 × 4_60 mm “phenomenex, Jupiter”. |
| Injection: | 20 μl. |
| Buffer A: | 80% 0.0125 M Tris, 0.0187 M (NH₄)₂SO₄ pH = 7, 20% |
| | CH₃CN. |
| Buffer B: | 80% CH₃CN, 20% water. |
| Flow: | 1.5 ml/min. |
| Gradient: | 0 min 5% B −> 20 min 55% B −> 22 min 80% B −> 24 min |
| | 80% B −> 25 min 5% B 32 min 5% B. |
| Detection: | UV 214 nm. |

Method 4:
Two Waters 510 HPLC pumps
Waters 2487 Dual λ Absorbance detector

| | |
|---|---|
| Column: | C4 5μ 150 × 4_60 mm “phenomenex, Jupiter” |
| Injection: | 20 μl |
| Buffer A: | 80% 0.0125 M Tris, 0.0187 M (NH₄)₂SO₄ pH = 7, 20% |
| | CH₃CN |
| Buffer B: | 80% CH₃CN, 20% water |
| Flow: | 1.5 ml/min |
| Gradient: | 0 min 10% B −> 20 min 50% B −> 22 min 60% B −> 23 min |
| | 10% B −> 30 min 10% B −> 31 min 10% B flow 0.15 min |
| Detection: | 214 nm |

Method 5:
Waters 2695 separations module
Waters 996 Photodiode Array Detector

| | |
|---|---|
| Column: | C4 5μ 150 × 4_60 mm “phenomenex, Jupiter” |
| Injection: | 25 μl |
| Buffer A: | 80% 0.01 M Tris, 0.015 M (NH₄)₂SO₄ pH = 7.3; 20% |
| | CH₃CN |
| Buffer B: | 20% water; 80% CH₃CN |
| Flow: | 1.5 ml/min |
| Gradient: | 1-20 min: 5-50% B, 20-22 min: 50-60% B, 22-23 min: |
| | 60-5% B, 23-30 min 5-0% B 30-31 min 0-5% B, flow: |
| | 0.15 ml/min. |
| Detection: | 214 nm |

Method 6:
Waters 2795 separations module
Waters 2996 Photodiode Array Detector
Waters Micromass ZQ 4000 electrospray mass spectrometer
LC-method:

| | |
|---|---|
| Column: | Phenomenex, Jupiter 5μ C4 300 Å 50 × 4.60 mm |
| Buffer A: | 0.1% TFA in water |
| Buffer B: | CH₃CN |
| Flow: | 1 ml/min |
| Gradient: | 0-7.5 min: 10-90% B |
| | 7.5-8.5 min: 90-10% B |
| | 8.5-9.5 min 10% B |
| | 9.5-10.00 min 10% B, flow: 0.1 ml/min |

MS method:

Mw: 500-2000 ES+
Cone Voltage 60 V
Scantime 1
InterScan delay: 0.1

Method 7:
Agilent 1100 series

| | |
|---|---|
| Column: | GraceVydac Protein C4, 5 um 4.6 × 250 mm (Cat# |
| | 214TP54) |
| Buffer A: | 10 mM Tris, 15 mM (NH₄)₂SO4, 20% CH₃CN in water |
| | pH 7.3 |
| Buffer B: | 20% water in CH3CN |
| Flow: | 1.5 ml/min |
| Gradient: | 1-20 min: 10% B to 50% B, 20-22 min: 50% B to 60% B, |
| | 22-23 min: 60% B to 10% B, 23-30 min 10% B 30-31 min |
| | 10% B, flow 0.15 ml/min. |
| Detection: | UV 214 nm |

### HPLC Method 8:

Anal. HPLC Waters:
Run time: 30 min

### Buffer A: 0.1% TFA in CH₃CN

### Buffer B: 0.1% TFA in MQ-water

Flow: 1.5 ml/min

### Gradient: 0-1 min: 10% B. 1-20 min: 10% B to 90% B. 20-22 min: 90% B. 22-23 min: 90% B to 10% B. 23-30 min: 10% B. 30-31 min 10% B

### Detection: UV 214 nm,

Column: C4 5μ 150×4_{—}60 mm “phenomenex, Jupiter”

### HPLC Method 9:

### Alliance 2696:

Run time: 30 min,
Buffer A: 80% 10 mM Tris, 15 mM (NH₄)₂SO₄ pH=7.3 20% CH₃CN,

### Buffer B: 80% CH₃CN, 20% MQ-water,

Flow: 1.5 ml/min,
Gradient: 0-2 min 0% B. 2-20 min: 0-70% B. 20-21 min: 70-0% B. 21-30 min: 0% B. 30 min-31 min 0% B.
Column: C4 5μ 150×4_{—}60 mm “phenomenex, Jupiter”

### Detection: UV 214 nm

### HPLC Method 10:

Alliance neut:
Run time: 30 min,
Eluents: A: 10 mM Tris, 15 mM (NH₄)₂SO₄, 20% CH₃CN i Mili Q water, pH 7.3

B: 20.0% MQ-water in CH₃CN.

Flow: 1.5 ml/min

### Gradient: 1-2 min: 0% B 2-20 min 0% B til 50% B. 20-21 min: 50-0% B. 22-30 min: 0% B. 30-31 min 0% B

### Detection: UV 214 nm

MALDI-TOF-MS spectra were recorded on a Bruker Autoflex II TOF/TOF operating in linear mode using a nitrogen laser and positive ion detection. Accelerating voltage: 20 kV.

### Preparation of Intermediates:

Myristic acid N-hydroxysuccinimide ester may be prepared according to B. FarouxCorlay et al., J. Med. Chem. 2001, 44, 2188-2203.

### Preparation of Hexadecandioyl-L-Glu(OSu)-OH:

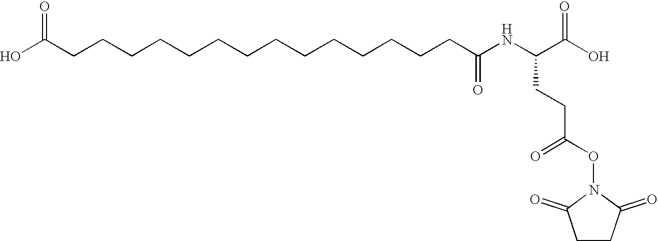

Hexadecanedioic acid (200 g, 0.7 mol) and Dowex50 WX2-100 ion exchange resin (700 g) was added n-octane (3.6 L) and the mixture was added benzyl formiate (95 g, 0.7 mol). The mixture was heated to 91° C. and more benzyl formiate (340 g, 2.5 mol) was added drop wise during 9 h. Heating at 91° C. was continued for 2 days and cooled to room temperature. The mixture was filtered and the solid was washed with n-octane (2.3 L) and dried by suction. The solid was suspended in acetone (3 L) and heated to 40° C. for 30 minutes. The mixture was filtered and the ion exchange resin was washed with acetone (1.5 L). The combined filtrates and washings (approx. 5 L) were concentrated by rotary evaporation to about 1.5 L. The formed suspension was filtered and the solid was washed with cold (−18° C.) acetone (0.8 L) and dried to afford 160 g crude material contaminated with unchanged hexadecanedioic acid. This was added dichloromethane (2 L) and Hyflo Super Cel Celite 545 (120 g) and the mixture was stirred for 30 minutes. The mixture was filtered and the solid was washed with dichloromethane (1 L). The combined filtrates and washings were evaporated in vacuo and the residue was recrystallised from 2-propanol (900 mL). This afforded 73 g (27.8%) of hexadecanedioic acid mono benzyl ester ¹H-NMR (DMSO-d₆): δ=1.23 (18H, s), 1.50 (4H, m), 2.18 (2H, t), 2.34 (2H, t), 5.08 (2H, s), 7.36 (5H, m), 12.0 (1H, bs).

Hexadecanedioic acid mono benzyl ester (71 g, 0.188 mol) was suspended in ethyl acetate (1 L) and N,N-diisopropylethylamine (34 g, 0.26 mol), N-hydroxysuccinimide (28.1 g, 0.24 mol), and 1-ethyl-3-(3′-dimethylaminopropyl)carbodiimide hydrochloride (46.8 g, 0.24 mol) were added successively and the mixture was stirred at room temperature for 5 days. The mixture was added ethyl acetate (600 mL) and was washed with aqueous potassium hydrogen sulphate (0.5M, 1 L). Saturated aqueous sodium chloride (700 mL) was added, and the phases were separated. The organic phase was successively washed with saturated aqueous sodium hydrogen carbonate (1 L) and saturated aqueous sodium chloride (800 mL). The organic phase was dried (MgSO₄) and evaporated in vacuo to afford 89.9 g of crude material. This was recrystallised from heptane (3.5 L) to afford 77.4 (87%) of hexadecanedioic acid benzyl ester N-hydroxysuccinimide ester. ¹H-NMR (DMSO-d₆): 1.23 (18H, m), 1.34 (2H, m), 1.53 (2H, m), 1.61 (2H, m), 2.34 (2H, t), 2.64 (2H, t), 2.80 (4H, s), 5.08 (2H, s), 7.35 (5H, m).

L-Glutamic acid α-benzyl ester (H-Glu-OBzl, 40.1 g, 0.17 mol) was suspended in N-methyl 2-pyrrolidinone (1 L). Triethylamine (28 mL, 0.2 mol) and hexadecanedioic acid benzyl ester N-hydroxysuccinimide ester (76.3 g, 0.16 mol) were added and the mixture was stirred at 50° C. for 2.5 hours. The mixture was added ethyl acetate (800 mL) and with cooling (internal temperature below 22° C.) was aqueous potassium hydrogen sulphate (0.5M, 800 mL) added in portions. The aqueous phase was extracted with ethyl acetate (400 mL). The combined organic phases were washed with saturated aqueous sodium chloride (3×600 mL), dried (MgSO₄) and concentrated in vacuo to afford 108.7 g (113%) of benzyl hexadecandioyl-L-Glu-OBzl as an oil containing some N-methyl 2-pyrrolidinone and ethyl acetate as seen from NMR. ¹H-NMR (DMSO-d₆), selected peaks: δ=1.22 (20H. s), 5.08 (2H, s), 5.11 (2H, s), 7.35 (1oH, m), 8.20 (1H, d), 12.2 (1H, bs).

Benzyl hexadecandioyl-L-Glu-OBzl (107 g, 0.16 mmol) was dissolved in ethyl acetate (1.1 L) and N,N-diisopropylethylamine (35 mL, 0.21 mol), N-hydroxysuccinimide (21.8 g, 0.19 mol), and 1-ethyl-3-(3′-dimethylaminopropyl)carbodiimide hydrochloride (39.3 g, 0.21 mol) were added successively and the mixture was stirred at room temperature for 16 hours. The mixture was added ethyl acetate (500 mL) and was washed with aqueous potassium hydrogen sulphate (0.5M, 0.75 L). Saturated aqueous sodium chloride (450 mL) was added, and the phases were separated. The organic phase was successively washed with saturated aqueous sodium hydrogen carbonate (450 mL) and saturated aqueous sodium chloride (2×450 mL). The organic phase was dried (MgSO₄) and concentrated in vacuo to about 500 mL. To the residue was added heptane (400 mL) and the mixture was stirred at room temperature for 16 hours. More heptane was added (800 mL) and the mixture was filtered and the solid was washed with a mixture of ethyl acetate and heptane (1:3, 50 mL) and dried to afford 97.6 g (89% over two steps) of benzyl hexadecandioyl-L-Glu(OSu)-OBzl. ¹H-NMR (DMSO-d₆): δ=1.22 (20H. s), 1.47-1.54 (4H, m), 2.09 (1H, m), 2.11 (3H, m), 2.33 (2H, t), 2.7-2.8 (2H, m), 2.81 (4H, s), 4.37 (1H, m), 5.08 (2H, s), 5.12 (2H, s), 7.35 (10H, m), 8.27 (1H, d).

Benzyl hexadecandioyl-L-Glu(OSu)-OBzl (99 g, 0.14 mol) was dissolved in acetone (1.9 L) containing trifluoroacetic acid (0.1%). The apparatus was flushed with nitrogen gas and palladium on carbon black (10%, dry, 19.8 g). The mixture was hydrogenated at room temperature and at atmospheric pressure (hydrogen consumption: 6.9 L). Under nitrogen, the mixture was filtered and the filtrate was added heptane (3 L) and cooled to 0-5° C. The mixture was filtered and the solid was washed with heptane (3×200 mL) and dried to afford 69.5 g (98%) of hexadecandioyl-L-Glu(OSu)-OH. ¹H-NMR (acetone-d₆, containing trifluoroacetic acid), selected peaks: δ=1.29 (20H, s), 1.4-1.6 (4H, m), 2.88 (4H, s), 4.61 (1H, m), 7.52 (2H, m).

### General Procedures for Acylation of Single-Chain Insulins of the Invention

General procedure (A)

Single-chain insulin (0.013 mmol) is dissolved in aqueous sodium carbonate (100 mM, 3.5 mL) and is added hexadecandioyl-L-Glu(OSu)-OH (16 mg, 0.032 mmol) dissolved in a mixture of N-methyl 2-pyrrolidinone (1 mL) and tetrahydrofuran (0.5 mL). Aqueous sodium hydroxide (1 N) is added to pH 11 and the resulting mixture is kept at room temperature for 45 minutes. More hexadecandioyl-L-Glu(OSu)-OH (16 mg, 0.032 mmol) dissolved in a mixture of N-methyl 2-pyrrolidinone (1 mL) and tetrahydrofuran (0.5 mL) is added and the resulting mixture is kept at room temperature for 1 hour. pH is adjusted to 5.6 with hydrochloric acid (1 N) and the mixture is centrifugated at 4000 rpm for 10 minutes and decanted. Purification by anion exchange chromatography and/or preparative HPLC followed by lyophilisation as indicated above affords the acylated compounds of the invention.

### General Procedure (B)

Alternatively, the acylation can be performed using a related tert-butyl-protected reagent followed by TFA-mediated deprotection of the intermediately protected acylated single-chain insulin, similarly as described in WO 2005012347 for two-chain insulins.

### General Procedure (C)

The single-chain insulin (0.016 mmol) is dissolved in aqueous sodium carbonate (100 mM, 2.2 mL) and added a solution of myristic acid N-hydroxysuccinimide ester (7.6 mg, 23 μmol, in a mixture of acetonitrile (1 mL) and tetrahydrofuran (0.6 mL). The resulting mixture is kept at room temperature for 40 minutes. If necessary, more myristic acid N-hydroxysuccinimide ester (3.8 mg in a mixture of acetonitrile (1 mL) and tetrahydrofuran (0.6 mL)) is added. The resulting mixture is kept at room temperature for 60 minutes. The mixture is diluted with water and lyophilized. Purification by anion exchange chromatography and/or preparative HPLC followed by lyophilisation as indicated above affords the acylated compounds of the invention.

### Recombinant Methods

All expressions plasmids are of the C—POT type, similar to those described in EP 171, 142, which are characterized by containing the Schizosaccharomyces pombe triose phosphate isomerase gene (POT) for the purpose of plasmid selection and stabilization in S. cerevisiae. The plasmids also contain the S. cerevisiae triose phosphate isomerase promoter and terminator. These sequences are similar to the corresponding sequences in plasmid pKFN1003 (described in WO 90/100075) as are all sequences except the sequence of the EcoRI-XbaI fragment encoding the fusion protein of the leader and the insulin product. In order to express different fusion proteins, the EcoRI-XbaI fragment of pKFN1003 is simply replaced by an EcoRI-XbaI fragment encoding the leader-insulin fusion of interest. Such EcoRI-XbaI fragments may be synthesized using synthetic oligonucleotides and PCR according to standard techniques.

Yeast transformants were prepared by transformation of the host strain S. cerevisiae strain MT663 (MATa/MATα pep4-3/pep4-3 HIS4/his4 tpi::LEU2/tpi::LEU2 Cir⁺). The yeast strain MT663 was deposited in the Deutsche Sammlung von Mikroorganismen und Zellkulturen in connection with filing WO 92/11378 and was given the deposit number DSM 6278.

MT663 was grown on YPGaL (1% Bacto yeast extract, 2% Bacto peptone, 2% galactose, 1% lactate) to an O.D. at 600 nm of 0.6. 100 ml of culture was harvested by centrifugation, washed with 10 ml of water, recentrifuged and resuspended in 10 ml of a solution containing 1.2 M sorbitol, 25 mM Na₂EDTA pH=8.0 and 6.7 mg/ml dithiotreitol. The suspension was incubated at 30° C. for 15 minutes, centrifuged and the cells resuspended in 10 ml of a solution containing 1.2 M sorbitol, 10 mM Na₂EDTA, 0.1 M sodium citrate, pH 0 5.8, and 2 mg Novozym®234. The suspension was incubated at 30° C. for 30 minutes, the cells collected by centrifugation, washed in 10 ml of 1.2 M sorbitol and 10 ml of CAS (1.2 M sorbitol, 10 mM CaCl₂, 10 mM Tris HCl (Tris=Tris(hydroxymethyl)aminomethane) pH=7.5) and resuspended in 2 ml of CAS. For transformation, 1 ml of CAS-suspended cells was mixed with approx. 0.1 mg of plasmid DNA and left at room temperature for 15 minutes. 1 ml of (20% polyethylene glycol 4000, 10 mM CaCl₂, 10 mM Tris HCl, pH=7.5) was added and the mixture left for a further 30 minutes at room temperature. The mixture was centrifuged and the pellet resuspended in 0.1 ml of SOS (1.2 M sorbitol, 33% v/v YPD, 6.7 mM CaCl₂) and incubated at 30° C. for 2 hours. The suspension was then centrifuged and the pellet resuspended in 0.5 ml of 1.2 M sorbitol. Then, 6 ml of top agar (the SC medium of Sherman et al. (1982) Methods in Yeast Genetics, Cold Spring Harbor Laboratory) containing 1.2 M sorbitol plus 2.5% agar) at 52° C. was added and the suspension poured on top of plates containing the same agar-solidified, sorbitol containing medium.

S. cerevisiae strain MT663 transformed with expression plasmids was grown in YPD for 72 h at 30° C.

### Example 1

### General Procedure (A)

### B(1-29)-B29A-VGLSSGQ-A(1-21)-A18K(N(eps)hexadecandioyl-gGlu) Human Insulin

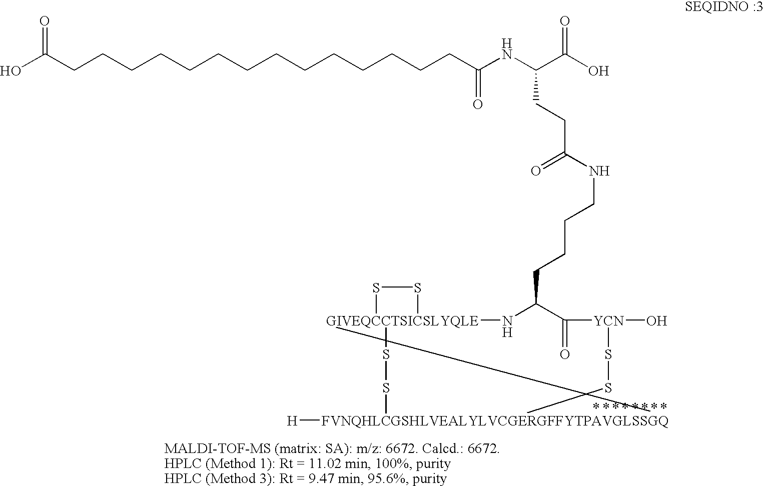

The insulin receptor binding measured according to assay (I) was 25% relative to that of human insulin.

### Example 2

### General Procedure (C)

### B(1-29)-B29A-VGLSSGQ-A(1-22)-A18Q, A22K(N(eps)myristoyl) Human Insulin

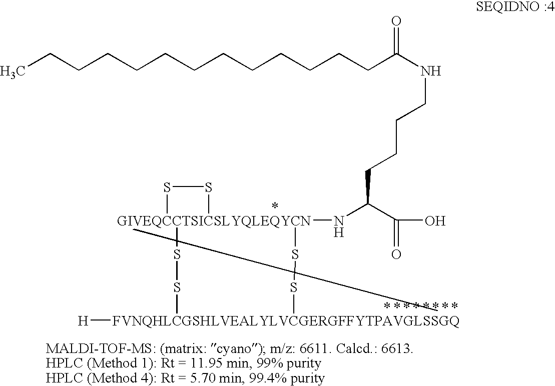

The insulin receptor binding measured according to assay (I) was 62% relative to that of human insulin.

### Example 3

### General Procedure (C)

### B(1-29)-B29A-VGLSSGQ-A(1-21)-A18K(N(eps)myristoyl) Human Insulin

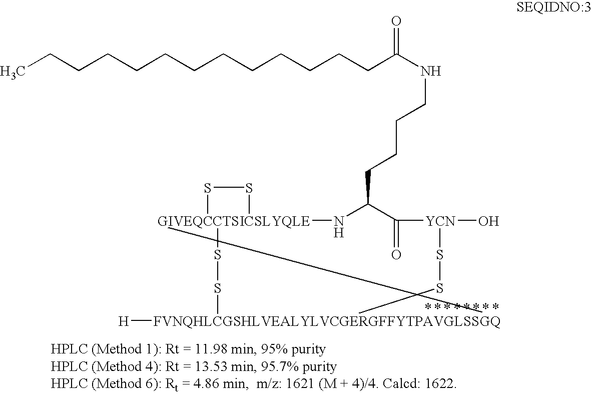

The insulin receptor binding measured according to assay (I) was 27% relative to that of human insulin.

### Example 4

### General Procedure (A)

### B(1-29)-B29A-VGLSSGQ-A(1-22)-A18Q, A22K(N(eps)hexadecandioyl-gGlu) Human Insulin

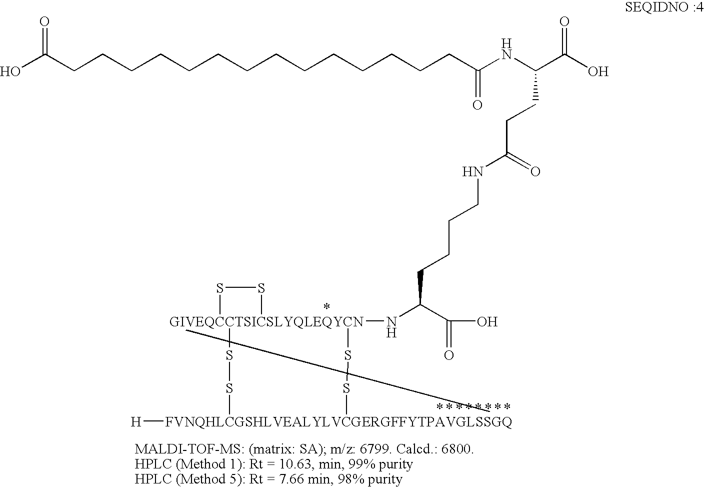

The insulin receptor binding measured according to assay (I) was 44% relative to that of human insulin.

### Example 5

### General Procedure (B)

### B(1-29)-B29A-VGLSSGQ-A(1-22)-A18Q, A22K(N(eps)octadecandioyl) Human Insulin

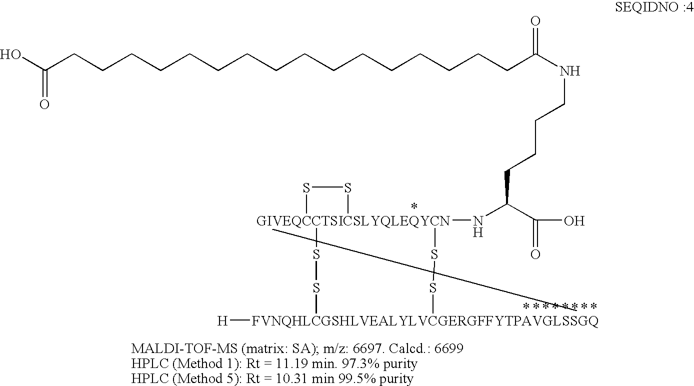

The insulin receptor binding measured according to assay (I) was 24% relative to that of human insulin.

### Example 6

### General Procedure (A)

B(1-29)-B29A-VGLSSGQ-A(1-21)-A8K(N(eps)hexadecandioyl-gGlu)-A18Q Human Insulin

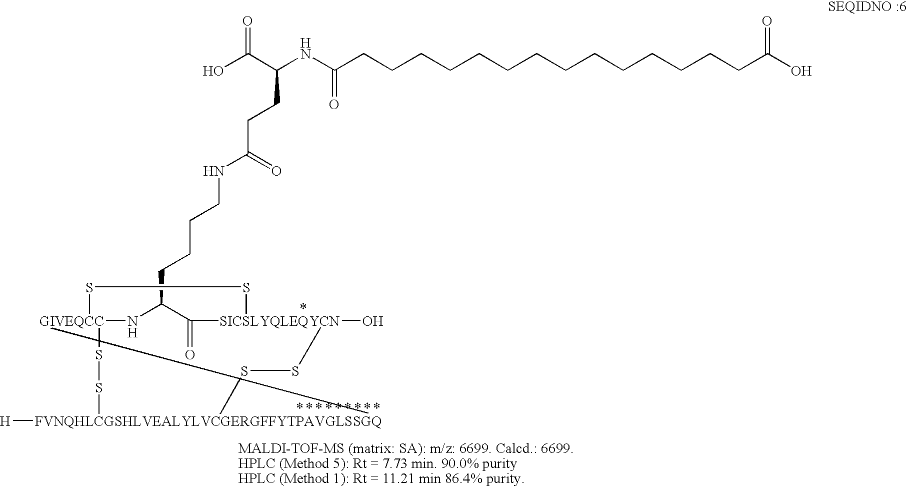

The insulin receptor binding measured according to assay (I) was 73% relative to that of human insulin.

### Example 7

### General Procedure (A)

B(1-29)-B29H-TGLGSGR-A(1-21)-A18K(N-eps-hexadecandioyl-g-L-Glu)-A21 G Human insulin

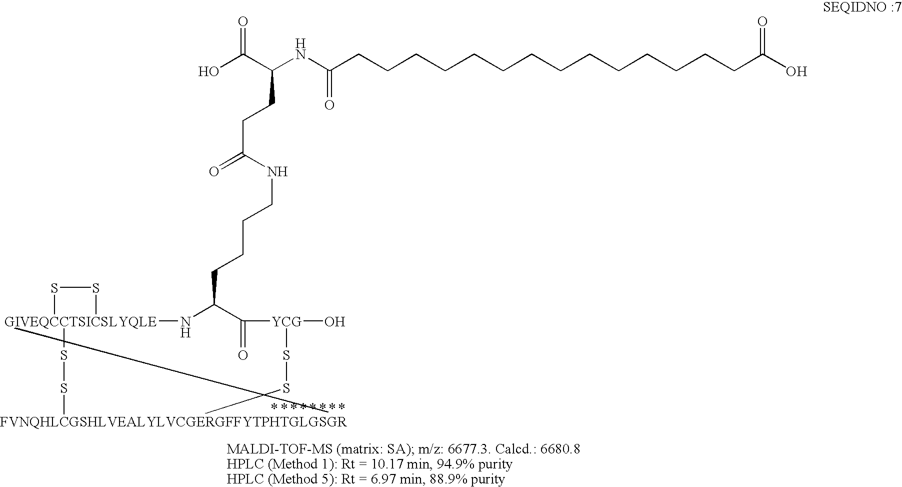

The insulin receptor binding measured according to assay (I) was 45.7% relative to that of human insulin.

### Example 8

### General Procedure (A)

B(1-29)-B29Q-TGLGSCR-A(1-22)-A18Q, A21 G-A22K(N (eps)hexadecand ioyl-g-LGlu) Human Insulin

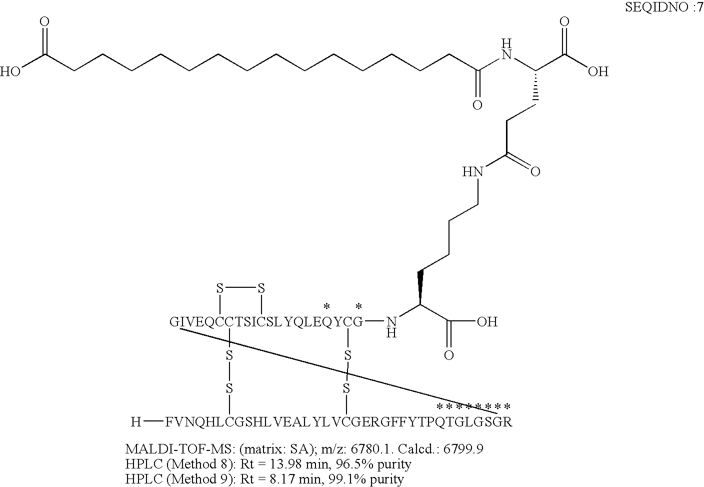

The insulin receptor binding measured according to assay (I) was 112.8% relative to that of human insulin.

### Example 9

### General Procedure (A)

B(1-29)-B29A-VGLSSGQ-A(1-21)-A15K(N(eps)-hexadecandioyl-g-L-Glu)-A18Q Human Insulin

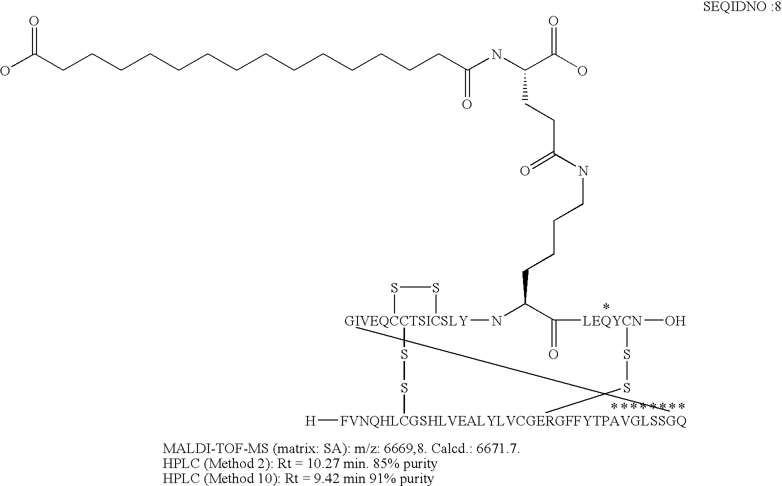

The insulin receptor binding measured according to assay (I) was 17.4% relative to that of human insulin.

### Example 10

### General Procedure (A)

B(1-29)-B29A-VGLSSGQ-A(1-22)-A18Q-A22K((eps)heptadecandioyl-g-Glu) Human Insulin

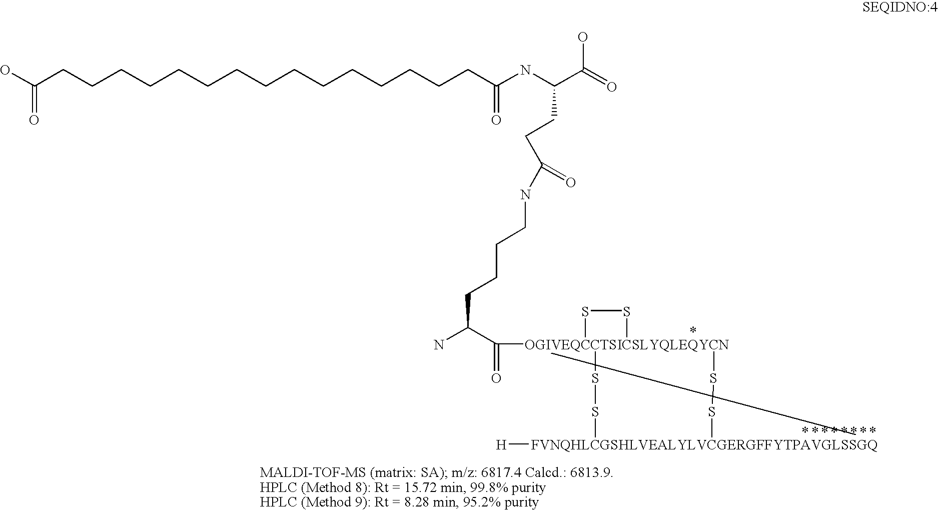

The insulin receptor binding measured according to assay (I) was 43% relative to that of human insulin.

### Pharmacological Methods

### Assay (I)

### Insulin Receptor Binding of the Acylated, Single-Chain Insulin

The affinity of the acylated, single-chain insulins for the human insulin receptor can be determined by a SPA assay (Scintillation Proximity Assay) microtiterplate antibody capture assay. SPA-PVT antibody-binding beads, anti-mouse reagent (Amersham Biosciences, Cat No. PRNQ0017) are mixed with 25 ml of binding buffer (100 mM HEPES pH 7.8; 100 mM sodium chloride, 10 mM MgSO₄, 0.025% Tween-20). Reagent mix for a single Packard Optiplate (Packard No. 6005190) is composed of 2.4 μl of a 1:5000 diluted purified recombinant human insulin receptor—exon 11, an amount of a stock solution of A14 Tyr[125I]-human insulin corresponding to 5000 cpm per 100 μl of reagent mix, 12 μl of a 1:1000 dilution of F12 antibody, 3 ml of SPA-beads and binding buffer to a total of 12 ml. A total of 100 μl is then added and a dilution series is made from appropriate samples. To the dilution series is then added 100 μl of reagent mix and the samples are incubated for 16 hours while gently shaken. The phases are then separated by centrifugation for 1 min and the plates counted in a Topcounter. The binding data are fitted using the nonlinear regression algorithm in the GraphPad Prism 2.01 (GraphPad Software, San Diego, Calif.).

### Assay (II)

### Potency of the Acylated, Single-Chain Insulins Relative to Human Insulin.

Wistar rats are used for testing the blood glucose lower efficacy of SCI af I.V bolus administration. Following administration the of either SCI or human insulin the concentration of blood glucose is monitored

### Assay (III)

### Determination in Pigs of T50% of the Acylated, Single-Chain Insulins

T50% is the time when 50% of an injected amount of the A14 Tyr[125I] labelled derivative of an insulin to be tested has disappeared from the injection site as measured with an external γ-counter.

The principles of laboratory animal care are followed, Specific pathogen-free LYYD, non-diabetic female pigs, cross-breed of Danish Landrace, Yorkshire and Duroc, are used (Holmenlund, Haarloev, Denmark) for pharmacokinetic and pharmacodynamic studies. The pigs are conscious, 4-5 months of age and weighing 70-95 kg. The animals are fasted overnight for 18 h before the experiment.

Formulated preparations of insulin derivatives labelled in TyrA14 with 125I are injected sc. in pigs as previously described (Ribel, U., Jørgensen, K, Brange, J. and Henriksen, U. The pig as a model for subcutaneous insulin absorption in man. Serrano-R10s, M and Lefèbvre, P. J. 891-896. 1985. Amsterdam; New York; Oxford, Elsevier Science Publishers. 1985 (Conference Proceeding)).

At the beginning of the experiments a dose of 60 nmol of the insulin derivative according to the invention (test compound) and a dose of 60 nmol of insulin (both 125I labelled in Tyr A14) are injected at two separate sites in the neck of each pig.

The disappearance of the radioactive label from the site of sc. Injection is monitored using a modification of the traditional external gamma-counting method (Ribel, U. Subcutaneous absorption of insulin analogues. Berger, M. and Gries, F. A. 70-77 (1993). Stuttgart; New York, Georg Thime Verlag (Conference Proceeding)). With this modified method it is possible to measure continuously the disappearance of radioactivity from a subcutaneous depot for several days using cordless portable device (Scancys Laboratorieteknik, Vaerløse, DK-3500, Denmark). The measurements are performed at 1-min intervals, and the counted values are corrected for background activity.

### Assay (IV)

### Pulmonary Delivery of Insulin Derivatives to Rats

The test substance will be dosed pulmonary by the drop instillation method. In brief, male Wistar rats (app.250 g) are anaesthetized in app. 60 ml fentanyl/dehydrodenzperidol/dormicum given as a 6.6 ml/kg sc primingdose and followed by 3 maintenance doses of 3.3 ml/kg sc with an interval of 30 min. Ten minutes after the induction of anaesthesia, basal samples are obtained from the tail vein (t=−20 min) followed by a basal sample immediately prior to the dosing of test substance (t=0). At t=0, the test substance is dosed intra tracheally into one lung. A special cannula with rounded ending is mounted on a syringe containing the 200 ul air and test substance (1 ml/kg). Via the orifice, the cannula is introduced into the trachea and is forwarded into one of the main bronchi-just passing the bifurcature. During the insertion, the neck is palpated from the exterior to assure intratracheal positioning. The content of the syringe is injected followed by 2 sec pause. Thereafter, the cannula is slowly drawn back. The rats are kept anaesthetized during the test (blood samples for up to 4 hrs) and are euthanized after the experiment.

1. An acylated, single-chain insulin comprising the B- and the A-chain of human insulin or an analogue thereof connected by a connecting peptide, wherein a lysine residue being substituted for the natural amino acid residue in one of the positions A12-A23 in the human insulin A-chain is chemically modified by acylation.

2. An acylated, single-chain insulin according to claim 1 being acylated at a lysine amino acid residue in position A12, A14, A15, A17, A18, A21, A22 or A23 in the human insulin A-chain.

3. An acylated, single-chain insulin according to claim 1 being acylated at a lysine amino acid residue in position A18, A21, A22 or A23 in the human insulin A-chain.

4. An acylated, single-chain insulin according to claim 1, wherein further the lysine amino acid residue in position B29 of the B-chain is acylated.

5. An acylated, single-chain insulin according to claim 1, wherein the natural lysine residue in position B29 of the B-chain of human insulin is substituted with another amino acid residue or is deleted.

6. An acylated, single-chain insulin according to claim 1, wherein the acyl group is a lipophilic group derived from a mono carboxylic or dicarboxylic, saturated or unsaturated, linear or branched fatty acid moiety having from about 6 to about 32 carbon atoms which may comprise at least one free carboxylic acid group or a group which is negatively charged at neutral pH.

7. An acylated, single chain insulin according to claim 6, wherein the fatty acid moiety has from 6 to 24, from 8 to 20, from 12 to 20, from 12-16, from 10-16, from 10-20, from 14-18 or from 14-16 carbon atoms.

8. An acylated, single chain insulin according to claim 1, wherein the acyl group is attached to the single-chain insulin by a linker molecule.

9. An acylated, single chain insulin according to claim 1, wherein the connecting peptide has from 3 to about 25, from 3 to about 20, from 4 to about 25, from 4 to about 20, from 5 to about 25, from 5 to about 20, from 6 to about 25, from 6 to about 20 amino, from 3 to about 15, from 3 to about 10, from 4 to about 15, from 4 to about 10, from 5 to about 15, from 5 to about 10, from 6 to about 15 or from 6 to about 10 amino acid residues in the peptide chain.

10. A pharmaceutical composition comprising an acylated, single chain insulin according to claim 1 together with the usual pharmaceutical adjuvants and additives.


SEQUENCE LISTING (8 sequences)

SEQ ID NO: 1 - protein, 7 aa, Artificial
  Connecting peptide
VGLSSGQ

SEQ ID NO: 2 - protein, 7 aa, Artificial
  Connecting peptide
TGLGSGR

SEQ ID NO: 3 - protein, 57 aa, Homo sapiens
  MOD_RES (54)..(54): ACETYLATION
FVNQHLCGSHLVEALYLVCGERGFFYTPAVGLSSGQGIVEQCCTSICSLYQLEKYCN

SEQ ID NO: 4 - protein, 58 aa, Homo sapiens
  MOD_RES (58)..(58): ACETYLATION
FVNQHLCGSHLVEALYLVCGERGFFYTPAVGLSSGQGIVEQCCTSICSLYQLEQYCNK

SEQ ID NO: 5 - protein, 57 aa, Homo sapiens
  MOD_RES (44)..(44): ACETYLATION
FVNQHLCGSHLVEALYLVCGERGFFYTPAVGLSSGQGIVEQCCKSICSLYQLEQYCN

SEQ ID NO: 6 - protein, 57 aa, Homo sapiens
FVNQHLCGSHLVEALYLVCGERGFFYTPHTGLGSGRGIVEQCCKSICSLYQLEKYCG

SEQ ID NO: 7 - protein, 58 aa, Homo sapiens
  MOD_RES (58)..(58): ACETYLATION
FVNQHLCGSHLVEALYLVCGERGFFYTPQTGLGSGRGIVEQCCKSICSLYQLEQYCGK

SEQ ID NO: 8 - protein, 57 aa, Homo sapiens
  MOD_RES (51)..(51): ACETYLATION
FVNQHLCGSHLVEALYLVCGERGFFYTPAVGLSSGQGIVEQCCKSICSLYKLEQYCN